SEQUENCE LISTING (3 sequences)

SEQ ID NO: 1 - protein, 431 aa, Artificial Sequence
  Aflibercept
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPG

SEQ ID NO: 2 - protein, 552 aa, Artificial Sequence
  conbercept
MVSYWDTGVLLCALLSCLLLTGSSSGGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKPFVAFGSGMESLVEATVGERVRLPAKYLGYPPPEIKWYKNGIPLESNHTIKAGHVLTIMEVSERDTGNYTVILTNPISKEKQSHVVSLVVYVPPGPGDKTHTCPLCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKATPPVLDSDGSFFLYSKLTVDKSRWQQGNVFSCSVMHEALHNHYTQKSLSLSPGK

SEQ ID NO: 3 - protein, 126 aa, Artificial Sequence
  DARPin MP0112
GSDLGKKLLEAARAGQDDEVRILMANGADVNTADSTGWTPLHLAVPWGHLEIVEVLLKYGADVNAKDFQGWTPLHLAAAIGHQEIVEVLLKNGADVNAQDKFGKTAFDISIDNGNEDLAEILQKAA

(19) United States
(12) Patent Application Publication
(10) Pub. No.: US 2015/0297675 A1
(43) Pub. Date: Oct. 22, 2015

(54) USE OF A VEGF ANTAGONIST IN TREATING OCULAR VASCULAR PROLIFERATIVE DISEASES

(71) Applicant: Aaron OSBORNE, Forth Worth, TX (US)

(72) Inventor: Aaron OSBORNE, Forth Worth, TX (US)

(21) Appl. No.: 14/423,719

(22) PCT Filed: Aug. 28, 2013
(86) PCT No.: PCT/EP2013/067846

§ 371 Date: Feb. 25, 2015

Related U.S. Application Data
(60) Provisional application No. 61/693,913, filed on Aug. 28, 2012.

(51) Int. Cl. A61K 38/17; A61K 45/06; A61F 9/008; A61K 9/00

(52) U.S. Cl. CPC A61K 38/179; A61K 9/0048; A61K 45/06; A61F 9/00821

(57) ABSTRACT

The present invention relates to the use of a non-antibody VEGF antagonist, in the treatment of choroidal neovascularisation secondary to diseases other than age-related macular degeneration and pathologic myopia.

This invention is in the field of treating retinal disorders. In particular, the present invention relates to the treatment of ocular vascular proliferative diseases.

### BACKGROUND ART

A lack of oxygen in the retina or cornea can result in the formation of new blood vessels—a process referred to as neovascularization. Low oxygen conditions induce the expression of vascular endothelial growth factor (VEGF) which stimulates the proliferation of new blood vessels.

Insufficient oxygen supply to the retina can be caused by several conditions. For example, in diabetic patients, insufficient insulin levels lead to an overaccumulation of glucose and/or fructose which in turn results in damage to the tiny blood vessels in the retina. This typically gives rise to abnormal blood vessel growth. In patients suffering from diabetic retinopathy, the disease often progresses from a non-proliferative stage to a proliferative stage. During the proliferative stage, fragile new blood vessels begin to extend into the clear, gel-like vitreous humour that fills the inside of the eye.

In other instances, oxygen supply is limited to parts of the retina due to the occlusion of retinal veins. The occlusion shuts off the blood supply to the areas downstream of it. Retinal vein occlusion is more prevalent in persons with high blood pressure, elevated cholesterol levels or diabetes. Retinal arteries may become thicker and stiff with age. Thickened arteries can compress the retinal vein either in the optic nerve (where they are located in close proximity to the retinal vein) or at points where the arteries cross the veins in the retina. Compression of retinal veins can ultimately lead to their occlusion—a condition called venous occlusive disease. Occlusion of the vein can occur, e.g., on the surface of the retina (branch retinal vein occlusion) or within the optic nerve (central retinal vein occlusion). The increased blood pressure in the veins also causes bleeding and swelling in the retina.

Ocular ischemic diseases such as diabetic retinopathy and central vein occlusion can lead to neovascular glaucoma. Neovascular glaucoma is a serious condition and is the result of elevated intraocular pressure (IOP) which may be due to the formation of abnormal neovascular tissue. These blood vessels may also appear on the surface of the iris—a condition referred to as rubeosis iridis.

Similarly, blood vessel ingrowth from the limbal vascular plexus into the cornea can occur when the cornea is continuously subjected to low oxygen conditions. Ischemia is one cause for corneal neovascularization. Persons who wear hydrogel contact lenses for long periods of time each day are at a particular risk to develop corneal neovascularization. However, other circumstances including infection, inflammation, trauma and loss of the limbal stem cell barrier can promote an environment that promotes VEGF release and can cause corneal neovascularization.

The VEGF-induced formation of new blood vessels is detrimental, and retinal, intertrabecular or corneal neovascularization can ultimately lead to vision loss. A recent small-scale clinical trial tested topical administration of ranibizumab (Lucentis®) and bevacizumab (Avastin®) in the treatment of corneal neovascularization. VEGF antagonists were found to be safe and effective treatments for corneal neovascularization when appropriate precautions are observed (Stevenson et al. Ocul Surf (2012) 10(2):67-83). Although direct comparisons were not conclusive, the results suggested that ranibizumab may be modestly superior to bevacizumab in terms of both onset of action and degree of efficacy.

Ghanem et al. (Middle East Afr J Ophthalmol (2009) 16(2):75-79) reported a clinical case series of sixteen patients with rubeosis iridis and secondary glaucoma who were administered an intravitreal injection of bevacizumab. This treatment led to regression of iris neovascularization with a subsequent drop of the IOP in eyes with neovascular glaucoma.

Another recent small-scale study evaluated the effects of panretinal laser photocoagulation therapy (LPT) compared with panretinal LPT plus intravitreal injection of 0.5 mg of ranibizumab in patients with high-risk proliferative diabetic retinopathy (Filho et al. Acta Ophthalmol (2011) 89(7):e567-72). The study found that intravitreal ranibizumab administered after panretinal LPT was associated with a larger reduction in leakage at week 48 compared with panretinal LPT alone. The adjunctive use of intravitreal ranibizumab appeared to protect against the modest visual acuity loss and the macular swelling usually observed in eyes that are treated with panretinal LPT alone.

Dastjerdi et al. (Arch Ophthalmol (2009) 127(4):381-389) report that topical bevacizumab leads to decrease in invasion area and vessel calibre and reduces the severity of corneal neovascularization without local or systemic side-effects.

Ocular vascular proliferative diseases can lead to permanent vision loss if left untreated. It is an object of the invention to provide further and improved treatments for ocular vascular proliferative diseases.

### DISCLOSURE OF THE INVENTION

The present invention relates to the use of a non-antibody VEGF antagonist in the treatment of ocular vascular proliferative diseases. The invention further provides treatment schedules that reduce the total number of doctor visits leading to greater patient compliance and better overall disease outcomes such as stabilization or improvement of visual acuity.

The invention also provides a non-antibody VEGF antagonist for use in a method for treating a patient having an ocular vascular proliferative disease, wherein said method comprises administering to the eye of a patient a non-antibody VEGF antagonist. The non-antibody VEGF antagonist may be administered intravitreally, e.g. through injection, or topically, e in form of eye drops.

The invention further provides the use of a non-antibody VEGF antagonist in the manufacture of a medicament for treating a patient having an ocular vascular proliferative disease.

Non-Antibody VEGF Antagonists

VEGF is a well-characterised signal protein which stimulates angiogenesis. Two antibody VEGF antagonists have been approved for human use, namely ranibizumab (Lucentis®) and bevacizumab (Avastin®). Patients suffering from ocular vascular proliferative diseases have been treated with bevacizumab and ranibizumab (Filho et al. Acta Ophthalmol (2011) 89(7):e567-72; Stevenson et al. Ocul Surf (2012) 10(2):67-83).

In one aspect of the invention, the non-antibody VEGF antagonist is an immunoadhesin. One such immuoadhesin is aflibercept (Eylea®), which has recently been approved for human use and is also known as VEGF-trap (Holash et al. (2002) PNAS USA 99:11393-98; Riely & Miller (2007) Clin Cancer Res 13:4623-7s). Aflibercept is the preferred non-antibody VEGF antagonist for use with the invention. Aflibercept is a recombinant human soluble VEGF receptor fusion protein consisting of portions of human VEGF receptors 1 and 2 extracellular domains fused to the Fc portion of human IgG1. It is a dimeric glycoprotein with a protein molecular weight of 97 kilodaltons (kDa) and contains glycosylation, constituting an additional 15% of the total molecular mass, resulting in a total molecular weight of 115 kDa. It is conveniently produced as a glycoprotein by expression in recombinant CHO K1 cells. Each monomer can have the following amino acid sequence (SEQ ID NO: 1):

SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKENSTEV
RVHEKDKTHTCPPCPAPELLGGPSVFLEPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVESCSVMHEALHNHYTQKSLSLSPG

and disulfide bridges can be formed between residues 30-79, 124-185, 246-306 and 352-410 within each monomer, and between residues 211-211 and 214-214 between the monomers.

Another non-antibody VEGF antagonist immunoadhesin currently in pre-clinical development is a recombinant human soluble VEGF receptor fusion protein similar to VEGF-trap containing extracellular ligand-binding domains 3 and 4 from VEGFR2/KDR, and domain 2 from VEGFR1/Flt-1; these domains are fused to a human IgG Fc protein fragment (Li et al., 2011 Molecular Vision 17:797-803). This antagonist binds to isoforms VEGF-A, VEGF-B and VEGF-C. The molecule is prepared using two different production processes resulting in different glycosylation patterns on the final proteins. The two glycoforms are referred to as KH902 (conbercept) and KH906. The fusion protein can have the following amino acid sequence (SEQ ID NO:2):

MVSYWDTGVLLCALLSCLLLTGSSSGGRPFVEMYSEIPEIIHMTEGRELV
IPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTAR
TELNVGIDFNWEYPSSKHQHKKLVNRDLKTQSGSEMKKELSTLTIDGVTR
SDQGLYTCAASSGLMTKKNSTEVRVHEKPEVAFGSGMESLVEATVGERVR
LPAKYLGYPPPEIKWYKNGIPLESNHTIKAGHVLTIMEVSERDTGNYTVI
LTNPISKEKQSHVVSLVVYVPPGPGDKTHTCPLCPAPELLGGPSVFLFPP
KPKDTLMISRTPEVTCVVVDVSHEDPEVKFNWYVDGVEVHNAKTKPREEQ
YNSTYRVVSVLTVLHQDWLNGKEYKCKVSNKALPAPIEKTISKAKGQPRE
PQVYTLPPSRDELTKNQVSLTCLVKGFYPSDIAVEWESNGQPENNYKATP
PVLDSDGSEELYSKLTVDKSRWQQGNVFSCSVMHEALHNHYTQKSLSLSP
GK

and, like VEGF-trap, can be present as a dimer. This fusion protein and related molecules are further characterized in EP1767546.

Other non-antibody VEGF antagonists include antibody mimetics (e.g. Affibody® molecules, affilins, affitins, anticalins, avimers, Kuntz domain peptides, and monobodies) with VEGF antagonist activity. This includes recombinant binding proteins comprising an ankyrin repeat domain that binds VEGF-A and prevents it from binding to VEGFR-2. One example for such a molecule is DARPin® MP0112. The ankyrin binding domain may have the following amino acid sequence (SEQ ID NO: 3):

GSDLGKKLLEAARAGQDDEVRILMANGADVNTADSTGWTPLHLAVPWGHL
EIVEVLLKYGADVNAKDFQGWTPLHLAAAIGHQEIVEVLLKNGADVNAQD
KFGKTAFDISIDNGNEDLAEILQKAA

Recombinant binding proteins comprising an ankyrin repeat domain that binds VEGF-A and prevents it from binding to VEGFR-2 are described in more detail in WO2010/060748 and WO2011/135067.

Further specific antibody mimetics with VEGF antagonist activity are the 40 kD pegylated anticalin PRS-050 and the monobody angiocept (CT-322).

The non-antibody VEGF antagonist may be modified to further improve its pharmacokinetic properties or bioavailability. For example, a non-antibody VEGF antagonist may be chemically modified (e.g., pegylated) to extend its in vivo half-life. Alternatively or in addition, it may be modified by glycosylation or the addition of further glycosylation sites not present in the protein sequence of the natural protein from which the VEGF antagonist was derived.

Variants of the above-specified VEGF antagonists that have improved characteristics for the desired application may be produced by the addition or deletion of amino acids. Ordinarily, these amino acid sequence variants will have an amino acid sequence having at least 60% amino acid sequence identity with the amino acid sequences of SEQ ID NO: 1, SEQ ID NO: 2 or SEQ ID NO: 3, preferably at least 80%, more preferably at least 85%, more preferably at least 90%, and most preferably at least 95%, including for example, 80%, 81%, 82%, 83%, 84%, 85%, 86%, 87%, 88%, 89%, 90%, 91%, 92%, 93%, 94%, 95%, 96%, 97%, 98%, 99%, and 100%. Identity or homology with respect to this sequence is defined herein as the percentage of amino acid residues in the candidate sequence that are identical with SEQ ID NO: 1, SEQ ID NO: 2 or SEQ ID NO: 3, after aligning the sequences and introducing gaps, if necessary, to achieve the maximum percent sequence identity, and not considering any conservative substitutions as part of the sequence identity.

Sequence identity can be determined by standard methods that are commonly used to compare the similarity in position of the amino acids of two polypeptides. Using a computer program such as BLAST or FASTA, two polypeptides are aligned for optimal matching of their respective amino acids (either along the full length of one or both sequences or along a pre-determined portion of one or both sequences). The programs provide a default opening penalty and a default gap penalty, and a scoring matrix such as PAM 250 [a standard scoring matrix; see Dayhoff et al., in Atlas of Protein Sequence and Structure, vol. 5, supp. 3 (1978)] can be used in conjunction with the computer program. For example, the percent identity can then be calculated as: the total number of identical matches multiplied by 100 and then divided by the sum of the length of the longer sequence within the matched span and the number of gaps introduced into the longer sequences in order to align the two sequences.

Non-antibody VEGF antagonists are preferred herein over antibody VEGF antagonists due their different pharmacokinetic profile when administered intravitreally. Preferably, the non-antibody VEGF antagonist of the invention binds to VEGF via one or more protein domain(s) that are not derived from the antigen-binding domain of an antibody. The non-antibody VEGF antagonist of the invention are preferably proteinaceous, but may include modifications that are non-proteinaceous (e.g., pegylation, glycosylation).

Patient

In one aspect of the invention, non-antibody VEGF antagonists are particularly useful for treating patients with ocular vascular proliferative diseases. A hallmark of ocular vascular proliferative diseases is the undesired proliferation of new blood vessels, often in places that are normally not vascularized, such as the cornea or iris. The proliferation of new blood vessels may be triggered by insufficient oxygen supply to the retina or cornea. Low oxygen conditions directly induce expression of VEGF and thus stimulate neovascularization.

The proliferation of new blood vessels in the retina, the iris, the intertrabecular spaces and the cornea leads to a variety of more or less distinct conditions such as proliferative diabetic retinopathy, venous occlusive disease (most commonly due to branch or central retinal vein occlusion), rubeosis iridis, and corneal neovascularization. Neovascular glaucoma may develop as a late complication of ischemic retinopathies such as diabetic retinopathy or central retinal vein occlusion. Corneal neovascularization may occur secondarily to infection or inflammation in the eye, trauma to the eye (including chemical burns), or loss of the limbal stem cell barrier. For example, patients suffering from herpetic keratitis, trachoma or onchocerciasis typically also suffer from corneal neovascularization. Wearers of contact lenses are at an increased risk of developing corneal neovascularization. Extended use of contact lenses (e.g. more than 12 hours per day) may lead to hypoxia and irritation of the eye triggering corneal inflammation. Contact lens contamination can also cause corneal inflammation. Choroidal haemangioma, a type of benign vascular tumour, can also be associated with the proliferation of new blood vessels.

Patients with any of the conditions mentioned in the previous paragraph will benefit from the use of non-antibody antagonist.

A patient's medical history is usually used to determine the underlying cause for the development of ocular vascular proliferative diseases. The medical history as well as previous treatments may inform specific treatment options, in particular for combination treatments.

For example, patients having received one or more rounds of laser treatment may particularly benefit from the non-antibody VEGF antagonist therapy of the present invention. Combining non-antibody VEGF antagonist therapy with laser therapy may reduce the total treatment time as well as adverse effects that are often observed with laser therapy alone.

For patients in whom the ocular vascular proliferative disease is triggered by an inflammatory response, combination therapy with an anti-inflammatory agent can be considered. For example, the combined use of steroids and non-antibody VEGF antagonist therapy to reduce inflammation and prevent formation of new blood vessels, respectively, may be particularly advantageous in patients with corneal neovascularization. Patients who suffer from corneal neovascularization secondary to bacterial, viral, fungal or acanthamoebal infection may benefit from non-antibody VEGF antagonist therapy in combination with an antimicrobial agent and optionally an anti-inflammatory agent.

Patients with corneal stromal blood vessels due to corneal neovascularization are at a significant risk for immune rejection after corneal transplantation. Use of non-antibody VEGF antagonist therapy prior to (and optionally also subsequent to) conical transplantation therefore may be particularly beneficial to patients with corneal stromal blood vessels as successful reduction of corneal vascularization will reduce the risk of graft rejection.

Patients who would require multiple intravitreal injections (e.g., more than 3 injections, preferably more than 6 injections) of a VEGF antagonist other than the non-antibody VEGF antagonist of the invention to manage their ocular vascular proliferative diseases will benefit in particular from the non-antibody therapies of the invention.

Administration

The non-antibody VEGF antagonist of the invention will generally be administered to the patient via intravitreal injection, though other routes of administration may be used, such as a slow-release depot, an ocular plug/reservoir or eye drops. Administration in aqueous form is usual, with a typical volume of 20-150 μl e.g. 40-60 μl, or 50 μl. Injection can be via a 30-gauge×½-inch (12.7 mm) needle. For example, aflibercept is generally administered via intravitreal injection at a dose of 2 mg (suspended in 0.05 mL buffer comprising 40 mg/mL in 10 mM sodium phosphate, 40 mM sodium chloride, 0.03% polysorbate 20, and 5% sucrose, pH 6.2). However, the normal dose may be reduced for the treatment of smaller children and in particular infants. The dose for treating an infant with a VEGF antagonist of the invention is usually 50% of the dose administered to an adult. Smaller doses (e.g., 0.5 mg per monthly injection) may also be used. Patients suffering from corneal neovascularization may particularly benefit from topical administration of the non-antibody VEGF antagonist in form of eye drops. Further preferred modes of administration for patients with corneal neovascularization are subconjunctival injection or intracorneal injection.

Alternatively, an intravitreal device is used to continuously deliver a non-antibody VEGF antagonist into the eye over a period of several months before needing to be refilled by injection. Various intravitreal delivery systems are known in the art. These delivery systems may be active or passive. For example, WO2010/088548 describes a delivery system having a rigid body using passive diffusion to deliver a therapeutic agent. WO2002/100318 discloses a delivery system having a flexible body that allows active administration via a pressure differential. Alternatively, active delivery can be achieved by implantable miniature pumps. An example for an intravitreal delivery system using a miniature pump to deliver a therapeutic agent is the Ophthalmic MicroPump System™ marketed by Replenish, Inc. which can be programmed to deliver a set amount of a therapeutic agent for a pre-determined number of times.

The non-antibody VEGF antagonist is typically encased in a small capsule-like container (e.g., a silicone elastomer cup). The container is usually implanted in the eye above the iris. The container comprises a release opening. Release of the non-antibody VEGF antagonist may be controlled by a membrane positioned between the non-antibody VEGF antagonist and the opening, or by means of a miniature pump connected to the container. Alternatively, the non-antibody VEGF antagonist may be deposited in a slow-release matrix that prevents rapid diffusion of the antagonist out of the container.

Preferably, the intravitreal device is designed to release the non-antibody VEGF antagonist at an initial rate that is higher in the first month. The release rate slowly decreases, e.g., over the course of the first month after implantation, to a rate that is about 50% less than the initial rate. The container may have a size that is sufficient to hold a supply of the non-antibody VEGF antagonist that lasts for about four to six months. Since a reduced dose of VEGF antagonist may be sufficient for effective treatment when administration is continuous, the supply in the container may last for one year or longer, preferably about two years, more preferably about three years.

Because only a small surgery is required to implant a delivery system and intravitreal injections are avoided, patient compliance issues with repeated intravitreal injections can be avoided. Intravitreal concentrations of the non-antibody VEGF antagonist are reduced, and therefore the potential risk of side-effects from non-antibody VEGF antagonist entering the circulation is decreased. This aspect may be of a particular advantage in children who may require general anaesthesia for intravitreal injections. Systemically elevated non-antibody VEGF antagonist levels may interfere with normal growth and development of children who therefore may benefit from lower intravitreal concentrations of the non-antibody VEGF antagonist.

In one aspect of the invention, the non-antibody VEGF antagonist is provided in a pre-filled sterile syringe ready for administration. Preferably, the syringe has low silicone content. More preferably, the syringe is silicone free. The syringe may be made of glass. Using a pre-filled syringe for delivery has the advantage that any contamination of the sterile antagonist solution prior to administration can be avoided. Pre-filled syringes also provide easier handling for the administering ophthalmologist.

Slow-Release Formulations

Non-antibody VEGF antagonist may be provided as slow-release formulations. Slow-release formulations are typically obtained by mixing a therapeutic agent with a biodegradable polymer or encapsulating it into microparticles. By varying the manufacture conditions of polymer-based delivery compositions, the release kinetic properties of the resulting compositions can be modulated.

A slow-release formulation in accordance with the invention typically comprises a non-antibody VEGF antagonist, a polymeric carrier, and a release modifier for modifying a release rate of the non-antibody VEGF antagonist from the polymeric carrier. The polymeric carrier usually comprises one or more biodegradable polymers or co-polymers or combinations thereof. For example, the polymeric carrier may be selected from poly-lactic acid (PLA), poly-glycolic acid (PGA), poly-lacticle-co-glycolide (PLGA), polyesters, poly (orthoester), poly(phosphazine), poly (phosphate ester), polycaprolactones, or a combination thereof. A preferred polymeric carrier is PLGA. The release modifier is typically a long chain fatty alcohol, preferably comprising from 10 to 40 carbon atoms. Commonly used release modifiers include capryl alcohol, pelargonic alcohol, capric alcohol, lauryl alcohol, myristyl alcohol, cetyl alcohol, palmitoleyl alcohol, stearyl alcohol, isostearyl alcohol, elaidyl alcohol, oleyl alcohol, linoleyl alcohol, polyunsaturated elaidolinoleyl alcohol, polyunsaturated linolenyl alcohol, elaidolinolenyl alcohol, polyunsaturated ricinoleyl alcohol, arachidyl alcohol, behenyl alcohol, erucyl alcohol, lignoceryl alcohol, ceryl alcohol, montanyl alcohol, cluytyl alcohol, myricyl alcohol, melissyl alcohol, and geddyl alcohol.

Preferably, the non-antibody VEGF antagonist is incorporated into a microsphere-based sustained release composition. The microspheres are preferably prepared from PLGA. The amount of non-antibody VEGF antagonist incorporated in the microspheres and the release rate of the non-antibody VEGF antagonist can be controlled by varying the conditions used for preparing the microspheres. Processes for producing such slow-release formulations are described in US 2005/0281861 and US 2008/0107694.

Treatment Regimens

In comparison to antibody VEGF antagonists, non-antibody VEGF antagonists of the invention allow increased spacing between administrations resulting in a more cost-effective therapy. In addition, better patient compliance is achieved when intravitreal injections have to be performed less frequently. This is particularly advantageous in patients suffering from ocular vascular proliferative diseases who may require multiple injections to improve visual acuity or prevent vision loss.

In some cases, a single injection of the VEGF antagonist according to the invention may be sufficient to ameliorate the disease or prevent disease progression for many years. In other cases, three injections each one month apart are administered to the patient, while any subsequent injections are performed less frequently or on an as-needed basis. In certain cases, two injections spaced 6 weeks apart, preferably 8 weeks apart, more preferably 10 weeks apart may be required to improve visual acuity or halt disease progression. In other cases, three or more injections may be needed. In these cases, the time between injections should be at least 6 weeks, preferably 8 weeks, more preferably 10 weeks apart. Treatment may be continued until maximum visual acuity is achieved. For example, treatment may be discontinued when visual acuity is stable for at least three months (i.e., no increase or decrease in visual acuity is observed during this period).

Some treatment regimens may include an extended loading period in which the patient receives five or six intravitreal injections of the VEGF antagonist. Each injection is administered at least 4 weeks apart (e.g. one month apart). After the loading period, injections can be continued every 4 weeks or every month, but more typically they will be administered less frequently, e.g. every 8 weeks or every two months.

Disease progression or recurrence of an ocular vascular proliferative disease may require one or more or continued treatment cycles. For example, in a first cycle, two injections spaced 6 weeks, preferably 8 weeks, more preferably 10 weeks apart may be administered followed by an interruption of treatment for 3 months, 4 months, 5 months, 6 months, 9 months, 12 months, 24 months or 36 months. If the ocular vascular proliferative disease reappears, the treatment is continued with a second cycle. In some cases three, four, five or more treatment cycles may be needed. For example, a further treatment cycle may be initiated if worsening of visual acuity is observed (e.g., by monthly checking a patient's vision after treatment has been discontinued).

In another aspect of the invention, the non-antibody VEGF antagonist according to the invention is administered as needed. The non-antibody VEGF antagonist is administered the first time after an initial diagnosis of ocular vascular proliferative diseases has been made. A diagnosis of an ocular vascular proliferative disease can be made during examination of the eye by a combination of slit-lamp evaluation and biomicroscopic fundus examination with optical coherence tomography (OCT) and/or fluorescein fundus angiography. A second, third or further administration of the non-antibody VEGF antagonist is performed only if examination of the eye reveals signs of persistent or recurring ocular vascular proliferative disease. Typically, best-corrected visual acuity (BCVA) letter score is recorded at baseline and each subsequent visit. Treatment may be continued or resumed if the patient has lost more than five letters of BCVA from baseline. Other retreatment criteria may include (i) an increase in central retinal thickness from the lowest central retinal thickness measurement as confirmed by OCT; (ii) the presence of new subretinal or intraretinal blood, or an increase of subretinal or intraretinal blood in comparison to the last visit; and/or (iii) additional neovascularisation as confirmed by fluorescein angiography.

Combination Therapy

The compounds of the invention may be used in combination with one or more additional treatment.

In one aspect of the invention, treatment with a VEGF antagonist of the invention may be performed in combination with laser photocoagulation therapy (LPT) and photodynamic therapy (PDT). Laser treatment in some cases can itself lead to choroidal neovascularization. Laser therapy including LPT or PDT preferably should not be used in patients who previously responded with choroidal neovascularization to laser therapy.

LPT uses laser light to cause controlled damage of the retina to produce a beneficial therapeutic effect. Small bursts of laser light can seal leaky blood vessels, destroy abnormal blood vessels, seal retinal tears, or destroy abnormal tissue in the back of the eye. It is quick, non-invasive, and usually requires no anaesthesia other than an anaesthetic eye drop. LPT techniques and apparatuses are readily available to ophthalmologists (see Lock et al. (2010) Med J Malaysia 65:88-94).

LPT techniques can be classified as focal, panretinal (or scatter), or grid. Focal LPT applies small-sized burns to specific areas of focal leakage (microaneurysms) in the macula, Panretinal LPT scatters burns throughout the peripheral fundus. Grid LPT applies a pattern of burns to macular areas arising from diffuse capillary leakage or non-perfusion, with each burn typically spaced apart by two visible burn widths. Patients can receive more than one type of LPT (e.g. a combination of focal and panretinal LPT) and these may be administered one directly after the other, or after a delay. A useful panretinal LPT involves 1200-1600 burns.

Laser spot sizes (spot diameters) of 50-500 μm are typical (smaller spot sizes are more usual for focal LPT, larger for panretinal), applied for 50-200 ms (continuously, or via micropulses), using green-to-yellow wavelengths e.g. using an argon gas (514.5 nm) laser, a frequency-doubled Nd-YAG (532 nm) laser, a krypton yellow laser (568.2 nm), or a tunable dye laser (variable wavelength). In some cases a red laser may be used if a green or yellow laser is precluded (e.g. if vitreous hemorrhage is present).

Micropulse laser therapy (MLP) uses 810 nm or 577 nm lasers to direct a discontinuous beam of laser light on the affected tissue (Kiire et al. (2011) Retina Today, 67-70). This results in a greater degree of control over the photothermal effects in laser photocoagulation. The steady continuous-wave emission of conventional LPT is delivered in the form of short laser pulses. Each pulse typically is 100-300 μs in length with a 1700 to 1900 μs interval between each pulse. The “width” (“ON” time) of each pulse and the interval between pulses (“OFF” time) are adjustable by the surgeon. A shorter micropulse “width” limits the time for the laser-induced heat to spread to adjacent tissue. A longer interval between pulses allows cooling to take place before the next pulse is delivered. Intraretinal damage thus can be avoided. Hence MLP is also referred to as “sub-threshold laser treatment” or “tissue-sparing laser therapy”. 10-25% of micropulse power is sufficient to show a consistent photothermal effect that is confined to the retinal pigment epithelium and does not affect the neurosensory retina.

In one aspect of the invention, patients receive both LPT and a non-antibody VEGF antagonist. The administration of LPT and of non-antibody VEGF antagonist administration occur within 12 months of each other, preferably within six months of each other, and ideally occur within one month or less of each other (e.g. within 10 days). The administration of LPT and non-antibody VEGF antagonist may occur on the same day.

Typically, non-antibody VEGF antagonist therapy is administered prior to LPT. LPT can take place promptly after non-antibody VEGF antagonist administration (e.g. within 2-20 days, typically within 3-10 days), or can take place after a longer delay (e.g. after at least three weeks, after at least four weeks, after at least eight weeks, after at least 12 weeks, or after at least 24 weeks). For example, treatment with non-antibody VEGF antagonist may be initiated at least 1 week, 2 weeks, 3 weeks, 4 weeks, 2 months, 3 months, 4 months, 5 months or 6 months before LPT. The non-antibody VEGF antagonist may be administered every 4 weeks, every 6 weeks, or every 8 weeks. Treatment may be continued at the same interval or extended intervals after LPT. Where the interval is extended, the period between administration of the non-antibody VEGF antagonist may increase by 50% or 100%. For example, if the initial interval was 4 weeks, the interval may be extended to 6 or 8 weeks. If a patient receives LPT more than 12 weeks after receiving the non-antibody VEGF antagonist, their eye(s) might no longer contain detectable levels of the non-antibody VEGF antagonist.

Alternatively, non-antibody VEGF antagonist therapy may be administered after LPT. For instance, the non-antibody VEGF antagonist is administered to the patient within 1-2 hours after LPT, typically within 60 minutes after completion of the first LPT session. The non-antibody VEGF antagonist may subsequently be administered every 4 weeks, every 6 weeks, every 8 weeks, every 12 weeks or preferably every 16 weeks.

Some embodiments involve more than one administration of LPT and/or of non-antibody VEGF antagonist. For instance, in one useful embodiment a patient receives in series (i) non-antibody VEGF antagonist (ii) at least one administration of LPT (iii) non-antibody VEGF antagonist. For example, the patient may receive an initial intravitreal injection of non-antibody VEGF antagonist; then, within 3-10 days of receiving the non-antibody VEGF antagonist, they receive focal photocoagulation; then, either on the same day as the focal photocoagulation, or later, but to be initiated within 14 days of receiving the non-antibody VEGF antagonist, they receive at least one sitting (e.g. up to three) of panretinal photocoagulation (if administered in more than one sitting, the panretinal photocoagulation should be completed within two to four weeks of receiving the non-antibody VEGF antagonist); and then, four weeks or a month after the initial injection, they receive a second injection of the non-antibody VEGF antagonist. This regimen may be continued with further doses of the non-antibody VEGF antagonist e.g. with a frequency of every one or two months. Preferably, administration of the non-antibody VEGF antagonist is every four weeks (monthly) for the first three months. Afterwards administration of the non-antibody VEGF antagonist is once every eight weeks.

Alternatively, a patient receives in series (i) an administration of LPT and (ii) at least one administration of a non-antibody VEGF antagonist. The patient may receive an initial intravitreal injection of non-antibody VEGF antagonist within 1 or 2 hours after panretinal photocoagulation therapy. Four to sixteen weeks later, intravitreal injection of non-antibody VEGF antagonist is repeated. In some instances, one or more additional injections are performed only if new vessel formation continues. The need for additional injections will be reassessed after an additional period of 4-16 weeks since the last injection. For example, a patient receives panretinal photocoagulation therapy and, within approximately 60 minutes of receiving the laser treatment, is administered a first intravitreal injection of a non-antibody VEGF antagonist in the treated eye. At weeks 16 and 32 after the first injection, a second injection and a third injection, respectively, are administered, if new vessels are detected, e.g. by clinical assessment, colour photography, fluorescein angiography or on gonioscopy, during follow-up examinations.

In another, preferred aspect of the invention, the non-antibody VEGF antagonist according to the invention is administered as needed. For example, after completion of the first LPT session, the treated eye may be re-evaluated by a combination of slit-lamp evaluation and biomicroscopic fundus examination with optical coherence tomography (OCT) and/or fluorescein fundus angiography. Re-evaluation may take place at 4 weeks, 6 weeks, 8 weeks, 12 weeks or 16 weeks after the first LPT session. Subsequent follow-up visits may take place at 4 weeks, 6 weeks, 8 weeks, 12 weeks or 16 weeks after the first re-evaluation. A second, third or further administration of the non-antibody VEGF antagonist is performed only if examination of the eye reveals signs of persistent or recurring neovascularization.

Laser-induced tissue damage can stimulate the release of pro-angiogenic factors. Combination therapy of a non-antibody VEGF antagonist and LPT is particularly suitable to treat high-risk proliferative diabetic retinopathy. It is also suitable for treating corneal neovascularization and may reduce complications such as corneal haemorrhage, corneal thinning, iris atrophy and necrotizing scleritis.

PDT uses a light-activated molecule to cause localised damage to neovascular endothelium, resulting in vessel occlusion. Light is delivered to the retina as a single circular spot via a fiber optic cable and a slit lamp, using a suitable ophthalmic magnification lens (laser treatment). The light-activated compound is injected into the circulation prior to the laser treatment, and damage is inflicted by photoactivation of the compound in the area afflicted by neovascularization. One commonly used light-activated compound is verteporfin (Visudyne®). Verteporfin is transported in the plasma primarily by lipoproteins. Once verteporfin is activated by light in the presence of oxygen, highly reactive, short-lived singlet oxygen and reactive oxygen radicals are generated which damages the endothelium surrounding blood vessels. Damaged endothelium is known to release procoagulant and vasoactive factors through the lipo-oxygenase (leukotriene) and cyclooxygenase (eicosanoids such as thromboxane) pathways, resulting in platelet aggregation, fibrin clot formation and vasoconstriction. Verteporfin appears to somewhat preferentially accumulate in neovasculature. The wavelength of the laser used for photoactivation of the light-activated compound may vary depending on the specific light-activated compound used. For example, 689 nm wavelength laser light delivery to the patient 15 minutes after the start of the 10-minute infusion with verteporfin may be used. Photoactivation is controlled by the total light dose delivered. Using verteporfin in the treatment of choroidal neovascularization by PDT, the recommended light dose is 50 J/cm² of neovascular lesion administered at an intensity of 600 mW/cm² over 83 seconds. Light dose, light intensity, ophthalmic lens magnification factor and zoom lens setting are important parameters for the appropriate delivery of light to the predetermined treatment spot during PDT and may need to be adapted depending on the laser system used for therapy.

Administration of the non-antibody VEGF antagonist is performed before or after photodynamic therapy. Typically, administration of the non-antibody VEGF antagonist and PDT will be performed on the same day (e.g. within 24 hours of one another). In one embodiment, treatment with non-antibody antagonist is started up to 48 hours before photodynamic therapy. Alternatively, treatment with non-antibody VEGF antagonist is initiated at least 1 week, 2, weeks, 3 weeks, 4 weeks, 2 months, 3 months, 4 months, 5 months or 6 months before PDT. The non-antibody VEGF antagonist may be administered every 4 weeks, every 6 weeks, or every 8 weeks. Treatment may be continued at the same interval or extended intervals after PDT. Where the interval is extended, the period between administration of the non-antibody VEGF antagonist may increase by 50% or 100%. For example, if the initial interval was 4 weeks, the interval may be extended to 6 or 8 weeks. Alternatively, non-antibody VEGF antagonist administration may be continuous, for example, if an intravitreal delivery system is used. The intravitreal device may be implanted prior to PDT. Alternatively, a single administration of non-antibody VEGF antagonist shortly before or after PDT may be sufficient to achieve the desired effect. For example, a single dose of non-antibody VEGF antagonist may be given on the day of the PDT.

PDT may be repeated as needed. Generally, it is not given more frequently than every 3 months. PDT may be repeated every 3 months. For example, treatment may be continued until there has been complete regression of polyps in the treated eye(s). Alternatively, PDT may be repeated less frequently, in particular if the non-antibody VEGF antagonist treatment is continued after PDT. For example, intervals between PDT may be extended to every 4 months, every 5 months, or every 6 months. Ideally, continued treatment with a non-antibody VEGF antagonist after PDT prevents recurrence of the ocular vascular proliferative disease.

Combining PDT with non-antibody VEGF antagonist therapy is particularly useful in treating choroidal haemangioma as the combined use of both therapies may increase treatment efficacy and, at the same time, decrease undesired collateral vessel development.

In a further aspect of the invention, treatment time and patient compliance is improved by using a non-antibody VEGF antagonist in combination with an anti-inflammatory agent. Administering the VEGF antagonist in combination with an anti-inflammatory agent can have synergistic effects depending on the underlying cause of neovascularization. Addition of an anti-inflammatory agent is particularly advantageous in corneal neovascularization secondary to an inflammatory disease or condition. Anti-inflammatory agents include steroids and NSAIDs. NSAIDs used in the treatment of ocular diseases include ketorolac, nepafenac and diclofenac. In some instances, the use of diclofenac is preferred. Corticosteroids used in treating ocular diseases include dexamethasone, prednisolone, fluorometholone and fluocinolone. Other steroids or derivatives thereof that may be used in combination with VEGF antagonist treatment include anecortave, which has angiostatic effects but acts by a different mechanism than the VEGF antagonists according to the invention. A preferred anti-inflammatory agent is triamcinolone. The anti-inflammatory agent may also be a TNF-α antagonist. For example, a TNF-α antibody may be administered in combination with a non-antibody VEGF antagonist. TNF-α antibodies, e.g. those sold under the trade names Humira®, Remicade®, Simponi® and Cimzia®, are well known in the art. Alternatively, a TNF-α non-antibody antagonist such as Enbrel® may be administered in combination with anon-antibody VEGF antagonist.

The anti-inflammatory agent may be administered at the same time as the non-antibody VEGF antagonist. The anti-inflammatory agent can be administered either systemically or locally. For example, the anti-inflammatory agent may be administered orally, topically, or, preferably, intravitreally. In a preferred embodiment, triamcinolone is administered intravitreally at the same time as the non-antibody VEGF antagonist of the invention.

In yet another aspect of the invention, the non-antibody VEGF antagonist is administered after administration of an antimicrobial agent. For example, the antimicrobial agent may be selected from azithromycin, gatifloxacin, ciprofloxacin, ofloxacin, norfloxacin, polymixin B+chloramphenicol, chloramphenicol, gentamicin, fluconazole, sulfacetamide, tobramycin, neomycin+polymixin B, and netilmicin. Azithromycin is typically used to treat patients suffering from trachoma. Alternatively, the antimicrobial agent may be selected from pyrimethamine, sulfadiazine and folinic acid or a combination thereof. Combination with pyrimethamine can be particularly advantageous in treating patients with neovascularization associated with toxoplasmosis. In some instances, combination treatment with broad-spectrum antiparasitic avermectin medicines such as ivermectin may be beneficial (e.g., in patients suffering from onchocerciasis). Patients suffering from herpes simplex virus-induced keratitis will benefit from combining antiviral treatment either in the form of topical therapy with trifluridine or oral administration of acyclovir or valacyclovir with non-antibody VEGF antagonist therapy.

General

The term “comprising” encompasses “including” as well as “consisting” e.g. a composition “comprising” X may consist exclusively of X or may include something additional e.g. X+Y.

The term “about” in relation to a numerical value x is optional and means, for example, x±10%.

### MODES FOR CARRYING OUT THE INVENTION

### Comparative Example 1

A total of 397 patients were enrolled in a clinical study to assess the efficacy and safety of intraocular injections of 0.3 mg or 0.5 mg ranibizumab in patients with macular edema following branch retinal vein occlusion (BRVO). Eligible patients were randomized 1:1:1 to receive monthly intraocular injections of 0.3 mg or 0.5 mg of ranibizumab or sham injections.

The primary efficacy outcome measure was mean change from baseline best-corrected visual acuity (BCVA) letter score at month 6. Secondary outcomes included other parameters of visual function and central foveal thickness.

Intraocular injections of 0.3 mg or 0.5 mg ranibizumab provided rapid, effective treatment for macular edema following BRVO with low rates of ocular and nonocular safety events. Mean (95% confidence interval [CI]) change from baseline BCVA letter score at month 6 was 16.6 (14.7-18.5) and 18.3 (16.0-20.6) in the 0.3 mg and 0.5 mg ranibizumab groups and 7.3 (5.1-9.5) in the sham group (P<0.0001 for each ranibizumab group vs. sham). The percentage of patients who gained ≧15 letters in BCVA at month 6 was 55.2% (0.3 mg) and 61.1% (0.5 mg) in the ranibizumab groups and 28.8% in the sham group P<0.0001 for each ranibizumab group vs. sham). At month 6, significantly more ranibizumab-treated patients (0.3 mg, 67.9%; 0.5 mg, 64.9%) had BCVA of ≧20/40 compared with sham patients (41.7%; P<0.0001 for each ranibizumab group vs. sham). At the same time point, central foveal thickness had decreased by a mean of 337 μm (0.3 mg) and 345 μm (0.5 mg) in the ranibizumab groups and 158 μm in the sham group (P<0.0001 for each ranibizumab group vs. sham). The median percent reduction in excess foveal thickness at month 6 was 97.0% and 97.6% in 0.3 mg and 0.5 mg groups and 27.9% in the sham group. More patients in the sham group (54.5%) received rescue grid laser compared with the 0.3 mg (18.7%) and 0.5 mg (19.8%) ranibizumab groups.

### Comparative Example 2

Forty patients were enrolled in a clinical study. Only patients with high-risk proliferative retinopathy without prior laser treatment or vitrectomy were included in the study. Patients were randomly assigned to receive panretinal photocoagulation (PRP) or PRP plus intravitreal VEGF antagonist therapy. PRP was administered in two sessions (weeks 0 and 2). Six to eight hundred 500-μm spots were performed per session. Intravitreal ranibizumab was administered at the end of the first laser session in the group receiving intravitreal VEGF antagonist therapy. At weeks 16 and 32, patients were re-evaluated. If active new vessels were detected by fluorescein angiography, the eye was retreated. Patients in the PRP/VEGF antagonist group received intravitreal ranibizumab. Patients in the PRP group received 500-μm additional spots per quadrant of active new vessels.

Best-corrected visual acuity (BCVA) was determined according to the methods used in the Early Treatment Diabetic Retinopathy Study (ETDRS). Fluorescein angiography was employed to measure fluorescein leakage (FLA). Optical coherence tomography (OCT) was used to assess central subfield macular thickness (CSMT).

Twenty-nine of 40 patients initially enrolled in the study completed the 48-week follow-up evaluation. At baseline, mean±SE FLA (mm²) was 9.0±1.3 and 11.7±1.3 (p=0.1502); BCVA (log MAR) was 0.31±0.05 and 0.27±0.06 (p=0.6645); and CSMT (μm) was 216.3±10.7 and 249.4±36.1 (p=0.3925), in the PRP group and PRP/VEGF antagonist group, respectively. There was a significant (p<0.05) FLA reduction at all study visits in both groups. The reduction observed in the PRP/VEGF antagonist group was significantly larger than that in the PRP group at week 48 (PRP=2.9±1.3 mm²; PRP/VEGF antagonist group=5.8±1.3 mm²; p=0.0291). Worsening of BCVA was observed at 16, 32 and 48 weeks after treatment in the PRP group (p<0.05), while no significant BCVA changes were observed in the PRP/VEGF antagonist group. A significant CSMT increase was observed in the PRP group at all study visits. In contrast, a significant decrease in CSMT was observed in the PRP/VEGF antagonist group at week 16, and no significant difference in CSMT from baseline was observed at weeks 32 and 48.

### Example 3

The inhibitiory effect of subconjunctival injection of KH902 on corneal neovascularization in a rat model was tested. Corneal neovascularization was induced by alkaline burn. The rats were randomly divided into four groups: (1) group 1 received a subconjunctival injection of KH902 (30 mg/mL); (2) group 2 received a subconjunctival injection of dexamethasone (1 mg/mL); (3) group 3 received a subconjunctival injection of the solvent used to inject KH902 in group 1; (4) group 4 received a subconjunctival injection of saline, which was used as the solvent for dexamethasone in group 2.

At the 28th day after the treatments, the area of corneal neovascularization and the average optical density value of VEGF immunohistochemical staining in the four groups were measured.

On the 28th day after molding, the area of corneal neovascularization was significantly smaller in group 1 than in groups 2-4 (P<0.05 or P<0.01). VEGF expression levels were also significantly lower in group 1 than in groups 2-4 (P<0.01). Hence subconjunctival injection of KH902 is more effective than dexamethasone in inhibiting corneal neovascularization in a rat alkaline burn model.

### Example 4

Twenty patients are enrolled in an open-label pilot study to assess the use of 2.0 mg intravitreally administered aflibercept in the treatment of proliferative diabetic retinopathy. All patients present with active proliferative retinopathy at the time of enrolment. Patients are randomised into two groups.

After an initial loading period, the first group receives intravitreal aflibercept every four weeks, while the second group receives intravitreal aflibercept every eight weeks. During the loading period, patients in both groups receive five intravitreal injections of aflibercept beginning at day 1, and then at weeks 4, 8, 12, and 16. Following the five initial injections, patients in the first group will continue to receive aflibercept intravitreally every 4 weeks, beginning at week 20, through week 48, while patients in the second group will receive aflibercept intravitreally every 8 weeks, beginning week 24, through week 48.

Patients in both arms will be followed up every 4 weeks until week 52. The primary endpoint of the study will be at week 52 and will assess the incidence and severity of adverse events of intravitreal aflibercept injection in the treatment of proliferative diabetic retinopathy.

Secondary outcome measures are (i) the mean change in the area of fluorescein leakage in mm² area of neovascularisation) compared to baseline; (ii) proportion of patients with complete regression of neovascularisation; (iii) the mean change in ETDRS BCVA from baseline; (iv) the proportion of subjects gaining >5 letters, >10 letters and >15 letters from baseline; (v) the proportion of subjects losing >5 letters from baseline; (vi) the mean change in retinal thickness from baseline as demonstrated by OCT imaging; (vii) the proportion of subjects without vitreous hemorrhage or pre-retinal haemorrhage; (viii) the proportion of subjects with complete avoidance of panretinal laser photocoagulation (PRP)/additional PRP; and (ix) the proportion of subjects with avoidance of vitrectomy.

### Example 5

Twenty patients are enrolled in an open-label pilot study to assess the use of 2.0 mg (0.05 ml) intravitreally administered aflibercept in the treatment of neovascular glaucoma (NVG). Patients are randomised into two groups. Patients in the first group will receive a single, intravitreal injection of aflibercept injection at baseline followed by standard of care (PRP). Patients in the second group will receive three intravitreal aflibercept injection (one at baseline followed by two additional injections at 4 weeks and 8 weeks). Additional injections will be spaced every 8 weeks apart. Both groups will be treated for a total of 52 weeks.

The primary endpoint of the study will be an assessment of the safety profile of repeated intravitreal aflibercept injections in patients with NVG by evaluating the incidence and severity of adverse events. Secondary outcome measures are (i) the rate and extent of resolution of neovasculariaation; (ii) the mean change in intraocular pressure (IOP); (iii) the proportion of patients losing >5 letters on visual acuity; (iv) the proportion of patients gaining ≦5 letters on visual acuity; (v) the mean change in visual acuity; (vi) the visual field; (vii) the average retinal nerve fiber layer and central macular thickness; (viii) the need for additional IOP lowering medications; and (ix) the need for surgical intervention in both arms during the 52-week period.

### Example 6

Twenty-four patients are enrolled in an open-label study evaluating the impact of repeat intravitreal injections of aflibercept on capillary non-perfusion in patients with proliferative retinopathy and/or macular edema secondary to proliferative diabetic retinopathy and central retinal venous occlusive disease. Patients are randomised into two groups. Patients in the first group will receive intravitreal aflibercept injections every month for the 12 month duration of the study. Patients in the second group will receive intravitreal aflibercept injections every month for the first 6 months, then every other month for the next 6 months. Retreatment criteria will allow for patients to be treated every month in the second 6 months if needed. Primary outcome measures include the mean change in capillary non-perfusion as assessed by the presence and amount of capillary non-perfusion measured by wide-angle angiography at baseline, month 3, month 6, and month 12.

### Example 7

Ten patients with corneal neovascularization in one or more quadrants crossing more than 0.5 mm over the limbus at the time of corneal transplantation are enrolled in a phase 1, prospective, randomised, open-label clinical trial. Patients are randomised into two groups. Patients in the first group will receive 2 mg (0.05 mL) aflibercept via subconjunctival injection in addition to standard of care treatment (steroids and cyclosporine). Patients will receive one injection four weeks (+/−1 week) prior to transplantation. They will receive a second injection at the conclusion of corneal transplantation. Patients may receive as-needed repeat injections (minimum of 30 days in between treatments) for recurrence of corneal neovascularization (defined as >1.0 mm crossing onto the cornea, past the limbus, or extension of vessels beyond previously documented extent) during the follow-up period. Patients in the first group will receive standard of care (steroids and cyclosporine) treatment only. The primary endpoint in this study is safety as defined by incidence and severity of adverse events in patients with corneal neovascularization undergoing corneal transplant.

It will be understood that the invention is described above by way of example only and modifications may be made whilst remaining within the scope and spirit of the invention.

1. A method for treating a patient having an ocular vascular proliferative disease, comprising administering to the patient a non-antibody VEGF antagonist.

2. The method of claim 1, wherein the patient suffers from proliferative diabetic retinopathy, venous occlusive disease, rubeosis iridis, corneal neovascularization, or neovascular glaucoma.

3. The method of claim 2, wherein corneal neovascularization is secondary to an inflammatory condition.

4. The method of claim 3, wherein the inflammatory condition is triggered by an infectious agent.

5. The method of claim 3, wherein the inflammatory condition is herpetic keratitis, trachoma or onchocerciasis.

6. The method of claim 2, wherein corneal neovascularization is secondary to contact lens use.

7. The method of claim 1, wherein the non-antibody VEGF antagonist is administered prior to corneal transplantation.

8. The method of claim 1, wherein the non-antibody VEGF antagonist is administered in the form of eye drops.

9. The method of claim 1 further comprising administering to the patient an anti-inflammatory agent.

10. The method of claim 1 further comprising administering to the patient in combination with an antimicrobial, an antiviral or an anthelmintic agent.

11. The method of claim 2, wherein venous occlusive disease is due to branch retinal vein occlusion or central retinal vein occlusion.

12. The method of claim 2, wherein the patient suffers from proliferative diabetic retinopathy and the non-antibody VEGF antagonist is administered in combination with laser photocoagulation therapy.

13. The method of claim 12, wherein the non-antibody VEGF antagonist is administered prior to laser photocoagulation therapy.

14. The method of claim 1, wherein the non-antibody antagonist is selected from a recombinant human soluble VEGF receptor fusion protein and a recombinant binding protein comprising an ankyrin repeat domain that binds VEGF-A.

15. The method of claim 14, wherein the patient has received more than three injections of a VEGF antagonist other than the non-antibody VEGF antagonist of claim 14.

16. The method of claim 15, wherein both the non-antibody VEGF antagonist and the anti-inflammatory compound are administered intravitreally.

17. A method for treating a patient having an ocular vascular proliferative disease comprising administering a non-antibody VEGF antagonist every 6 weeks, every 8 weeks or every 10 weeks.

18. The method of claim 1, wherein the non-antibody VEGF antagonist is administered continuously.

19. A method for treating a patient having an ocular vascular proliferative disease comprising administering a first dose of a non-antibody VEGF antagonist after the initial diagnosis of the ocular vascular proliferative disease and wherein a second dose of the non-antibody VEGF antagonist is administered only if the ocular vascular proliferative disease persists or recurs after administration of the first dose.

20. The method of claim 19, wherein the interval between the first and the second treatment is at least 6 weeks, at least 8 weeks or at least 10 weeks.

21. The method of claim 19, wherein the interval between the first and the second treatment is at least 3 months, 6 month or 9 months.
